(19) United States
(12) Patent Application Publication
(10) Pub. No.: US 2025/0136538 A1
(43) Pub. Date: May 1, 2025

(54) ZINC CARBOXYLATE SALT FOR PRODUCTION OF SEMICONDUCTOR NANOPARTICLE

(71) Applicant: NOF CORPORATION, Tokyo (JP)

(72) Inventors: Takeshi Yoshimura, Amagasaki-shi (JP); Mako Nakamura, Amagasaki-shi (JP); Yuko Mitsuka, Tosu-shi (JP); Hirokazu Sasaki, Tosu-shi (JP); Takafumi Moriyama, Tosu-shi (JP); Makoto Kido, Tosu-shi (JP)

(73) Assignee: NOF CORPORATION, Tokyo (JP)

(21) Appl. No.: 18/683,994

(22) PCT Filed: Aug. 5, 2022
(86) PCT No.: PCT/JP2022/030043

§ 371 Date: Feb. 15, 2024

(30) Foreign Application Priority Data
Aug. 18, 2021 (JP) 2021-133632

(51) Int. Cl. C07C 53/128; B82Y 20/00; B82Y 40/00; C07C 53/126; C09K 11/08; C09K 11/88

(52) U.S. Cl. CPC C07C 53/128; C07C 53/126; C09K 11/0883; C09K 11/883; B82Y 20/00; B82Y 40/00

(57) ABSTRACT

A zinc carboxylate salt used in production of a semiconductor nanoparticle, in which a ratio of carboxylic acids having 8 to 10 carbon atoms in whole of carboxylic acids that form the zinc carboxylate salt is 80.0% by mass or more, and an average branching degree of the whole of the carboxylic acids that form the zinc carboxylate salt is in the range of 1.1 to 2.9. The present invention can provide a zinc salt used for easily producing a semiconductor nanoparticle having a core/shell type structure that has excellent optical properties in the case where two or more types of shell precursors are used to produce the semiconductor nanoparticle having a core/shell type structure.

### TECHNICAL FIELD

The present invention relates to a zine carboxylate salt for production of a semiconductor nanoparticle used in production of a core/shell type semiconductor nanoparticle.

### BACKGROUND ART

Semiconductor nanoparticles having a very fine particle size (quantum dot: QD) are used as a wavelength conversion material in a display. The semiconductor nanoparticle is a fine particle that can exhibit a quantum confinement effect, in which the width of the band gap varies depending on the size of the nanoparticle. The exciton formed in the semiconductor particle by a photoexcitation, a charge injection, or other means emits a photon having the energy corresponding to a band gap through recombination, making it possible to control the emission wavelength by adjusting the crystal size of the semiconductor nanoparticle, thereby enabling light having an intended wavelength to be emitted.

Currently, the semiconductor nanoparticle having a core/shell type structure is widely used as the semiconductor nanoparticle. This is because the core/shell type structure fills the dangling bond on the core surface, thereby reducing a surface defect.

The semiconductor nanoparticles that are used are those having such a core/shell type structure consisting of a group III-V type core and a group II-VI type shell. However, the difference in lattice constants between the group III-V type core and the group II-VI type shell can readily form a defect level, resulting in non-emission recombination of excitons through the defect level, thereby having a tendency to deteriorate the optical properties in the semiconductor nanoparticle formed through the defect level. Therefore, it is important to form the group II-VI type shell having generation of the defect level suppressed on the surface of the group III-V type core.

The SILAR method is known as the method for forming a shell on the surface of a core particle. In the SILAR method, shell precursors are alternately added to the core particle, and the added shell precursors react on the particle surface to form a shell. For example, a Zn precursor is first added to a core particle, followed by successively adding a S precursor, a Zn precursor, a S precursor, and so on, whereby alternately bringing the core particle into contact with the two types of shell precursors, i.e., the raw materials for the shell, to form alternating layers of the two types of shell precursors on the particle surface, in which the shell is formed by reacting these two types of shell precursors.

In addition, in Patent Literature 1 there is a description about the use of zinc oleate, zinc hexanoate, zinc octanoate, zinc laurate, zinc palmitate, zinc stearate, zinc dithiocarbamate, and the like as the Zn precursor to form the shell.

### CITATION LIST

### Patent Literature

Patent Literature 1: Japanese Translation of International Patent Application No. 2019-515338

### Non Patent Literature

Non-Patent Literature 1: Renguo Xie et al., J. AM. CHEM. SOC., 127, 7480-7488 (2005)
Non-Patent Literature 2: Ken-Tye Yong et al., ACS Nano, 3, 502-510 (2009)

### SUMMARY OF INVENTION

### Technical Problem

However, the SILAR method requires strict control of the amounts of the precursors added in the formation of the precursor layers; if the amounts of the precursors added are too small, the shell cannot be sufficiently formed, resulting in deterioration of optical properties, on the other hand, if the amounts are too much, the excess precursors may change particle's properties as well as this may cause formation of byproducts.

In addition, the zinc linear chain carboxylate salt described in Patent Literature 1 does not form a sufficient shell even used as a Zn precursor, so that it is insufficient to obtain excellent optical properties. In addition, because the core is contacted with the two types of shell precursors separately and alternately for multiple times, the method for production of the semiconductor nanoparticle becomes cumbersome.

Therefore, an object of the present invention is, in the case where two or more types of shell precursors are used to produce the semiconductor nanoparticle having a core/shell type structure, to provide a zinc salt to be used for simply producing a semiconductor nanoparticle having a core/shell type structure that has excellent optical properties.

### Solution to Problem

The inventors of the present invention carried out an extensive investigation to solve the above problem, and as a result, it has been found that by using a zinc salt of a carboxylic acid having a specific carbon number and a specific branching degree as a group II element precursor to be added to a core particle dispersion solution so as to react with a group VI element precursor on a surface of a core particle, a core/shell type semiconductor nanoparticle having excellent optical properties can be obtained even if the group II element precursor and the group IV element precursor are not brought into contact with the core particle separately and alternately multiple times, in other words, even if all the group II element precursor and the group VI element precursor are brought into contact and reacted with the core particle at a time, thereby completing the present invention.

Namely, the present invention (1) provides a zinc carboxylate salt used in production of a semiconductor nanoparticle, in which
a ratio of carboxylic acids having 8 to 10 carbon atoms in whole of the carboxylic acids that form the zinc carboxylate salt is 80.0% by mass or more, and
an average branching degree of whole of the carboxylic acids that form the zinc carboxylate salt is in the range of 1.1 to 2.9.

In addition, the present invention (2) provides the zinc carboxylate salt used for production of the semiconductor nanoparticle according to (1), in which the average branching degree in the whole of the carboxylic acids that form the zinc carboxylate salt is 1.3 to 2.7.

In addition, the present invention (3) provides the zinc carboxylate salt for production of the semiconductor nanoparticle according to (1) or (2), in which a ratio of carboxylic acids having 8 to 10 carbon atoms in the whole of the carboxylic acids that form the zinc carboxylate salt is 85.0% by mass or more.

In addition, the present invention (4) provides the zine carboxylate salt for production of the semiconductor nanoparticle according to any of (1) to (3), in which a rate of change in viscosity of the zinc carboxylate salt indicated by the following formula (1) is in a range of 95.0 to 100.0%:

$$\begin{matrix}General formula ( 1 ) & \\ Rate of change in viscosity ( \% ) = ( ( viscosity ( Pa * s ) at 130 °C - viscosity ( Pa * s ) at 50 °C ) / viscosity ( Pa * s ) at 50 °C ) \times 100 & ( 1 )\end{matrix}$$

(In the formula, the viscosity (Pa*s) at 130° C. is the value obtained by measuring the zinc carboxylate salt at a temperature of 130° C. by a dynamic viscoelasticity analyzer, and the viscosity at 50° C. is the value obtained by measuring the zinc carboxylate salt at a temperature of 50° C. by a dynamic viscoelasticity analyzer.)

### Advantageous Effect of Invention

According to the present invention, it is possible to provide a zine carboxylate salt to be used for simply producing a semiconductor nanoparticle having a core/shell type structure that has excellent optical properties in the case where two or more types of shell precursors are used to produce the semiconductor nanoparticle having a core/shell type structure.

### DESCRIPTION OF EMBODIMENTS

A zinc carboxylate salt according to the present invention is a zinc carboxylate salt used for production of a semiconductor nanoparticle, in which
a ratio of carboxylic acids having 8 to 10 carbon atoms in whole of the carboxylic acids that form the zinc carboxylate salt is 80.0% by mass or more, and
an average branching degree of the whole of the carboxylic acids that form the zinc carboxylate salt is in the range of 1.1 to 2.9.

The zine carboxylate salt according to the present invention is used to produce the semiconductor nanoparticle. Namely, the zinc carboxylate salt according to the present invention is used as a raw material zine carboxylate salt used in a method for producing the semiconductor nanoparticle, in which the method has a shell-forming process (hereinafter, shell-forming process (1)) at which a raw material zine carboxylate salt and a group VI element precursor are added to a core particle dispersion solution to form a shell that includes zinc and a group VI element on a surface of the core particle by reacting the raw material zinc carboxylate salt with the group VI element precursor in the presence of a core particle.

Hereinafter, the numerical range includes both ends unless otherwise specifically noted. In other words, the range of xx to yy means xx or more and yy or less.

At the shell-forming process (1), there is no particular restriction on the core particle on which a shell layer is formed as far as the particle is a particle that can be used as the core particle for the core/shell type semiconductor nanoparticle. Here, the core particle comprising In and P is preferable, and the core particle comprising In, P, and a halogen is especially preferable. It is preferable for the core particle to comprise In and P from the viewpoint of obtaining the semiconductor nanoparticle imposing a low environmental burden and having excellent optical properties. It is preferable that the core particle includes a halogen because it can improve the optical properties of the core particle and the semiconductor nanoparticle. The halogen that can be included in the core particle is F, Cl, Br, or I. Among these halogens, Cl and Br are preferable in view of a narrower full width at half maximum. The core particle may also include other elements such as Ga, Al, Zn, N, S, Si, and Ge.

The Cd content in the core particle is 100 ppm by mass or less, preferably 80 ppm by mass or less, and especially preferably 50 ppm by mass or less.

There is no particular restriction on the average diameter of the core particle; but it is preferable in the range of 1.0 nm to 5.0 nm. The average particle diameter of the core particle in the above range can make it possible to convert the excitation light of 450 nm into a green to red luminescence. In the present invention, the average particle diameter of the core particle is determined by calculating the area circular equivalent diameter (Heywood diameter) of 10 or more particles on a particle image observed by a transmission electron microscopy (TEM).

There is no particular restriction on the method for synthesizing the core particle; therefore, the method is chosen as appropriate. In the present invention, an In precursor, a P precursor, and a halogen precursor are as follows.

There is no particular restriction on the In precursor. Illustrative examples thereof include indium carboxylates such as indium acetate, indium propionate, indium myristate, and indium oleate; indium halides such as indium fluoride, indium bromide, and indium iodide; indium thiolate; and a trialkylindium.

There is no particular restriction on the P precursor. Illustrative examples thereof include tris(trimethylsilyl)phosphine, tris(trimethylgermyl)phosphine, tris(dimethylamino)phosphine, tris(diethylamino)phosphine, tris(dioctylamino)phosphine, a trialkyl phosphine, and a PH3 gas. When tris(trimethylsilyl)phosphine is used as the P precursor, there is the case that Si is incorporated into the semiconductor nanoparticle, but this does not impair the action of the present invention.

There is no particular restriction on the halogen precursor. Illustrative examples thereof include: HF, HCl, HBr, and HI; carboxylic acid halides such as oleyl chloride, oleyl bromide, octanoyl chloride, octanoyl bromide, and oleoyl chloride; and metal halides such as zinc chloride, indium chloride, and gallium chloride.

The following methods are used to synthesize the core particle comprising In and P. It should be noted, however, that the following methods of synthesizing the core particle are mere examples; and thus, the core particles are not limited to those synthesized by the following synthesis methods. The core particle may be synthesized, for example, by reacting an In precursor with a P precursor. First, an In precursor and a solvent are mixed, and the resulting In precursor solution, added with a dispersant and/or an additive as needed, is mixed under vacuum or a nitrogen atmosphere. After this is once heated at 100 to 300° C. for a period of 6 to 24 hours, a P precursor is added, which is followed by heating at 200 to 400° C. for a period of a few seconds (for example, 2 or 3 seconds) to 60 minutes, and then, cooling; and with this, the core particle dispersion solution in which the core particles are dispersed can be obtained. Next, a halogen precursor is added to the core particle dispersion solution, and then, the resulting mixture is heated at 25 to 350° C. for a period of a few seconds (for example, 2 or 3 seconds) to 60 minutes, and then this is cooled to obtain a halogen-doped core particle dispersion solution having the halogen partially on the surface of the core particle.

There is no particular restriction on the dispersant for this. Illustrative examples thereof include a carboxylic acid, an amine, a thiol, a phosphine, a phosphine oxide, a phosphine, and a phosphonic acid. The dispersant can also serve as a solvent. There is no particular restriction on the solvent. Illustrative examples thereof include 1-octadecene, hexadecane, squalane, oleylamine, trioctylphosphine, and trioctylphosphine oxide. Illustrative examples of the additive include a S precursor, a Zn precursor, and a halogen precursor, those having been described above.

The core particle dispersion solution for the shell-forming process (1) is the dispersion solution in which the core particles are dispersed in a dispersing medium. There is no particular restriction on the dispersing medium in which the core particles are dispersed. Illustrative examples thereof include 1-octadecene, hexadecane, squalane, squalene, mineral spirit, liquid paraffin, trioctylamine, trioctylphosphine, trioctylphosphine oxide, toluene, hexane, and diphenyl ether. These may be used singly or in a combination of two or more of them, and preferably at least one medium selected from the group consisting of 1-octadecene, hexadecane, squalane, squalene, mineral spirit, and liquid paraffin.

The raw material zinc carboxylate for the shell-forming process (1) is the zinc carboxylate salt according to the present invention, and the zine precursor that is added to the core particle dispersion solution, and the zinc precursor that reacts with the VI element precursor at the shell-forming process (1).

Of the whole of the carboxylic acids that form the zinc carboxylate salt according to the present invention, the ratio of the carboxylic acids having 8 to 10 carbon atoms is 80.0% by mass or more, preferably 85.0% by mass or more, more preferably 90.0% by mass or more, and especially preferably 100.0% by mass. In other words, the ratio of the carboxylic acids having 8 to 10 carbon atoms occupying the whole of the carboxylic acids that form the zine carboxylate salt according to the present invention is 80.0% by mass or more, preferably 85.0% by mass or more, more preferably 90.0% by mass or more, and especially preferably 100.0% by mass. The semiconductor nanoparticle having excellent optical properties can be obtained when the ratio of the carboxylic acids having 8 to 10 carbon atoms in the whole of the carboxylic acids that form the zinc carboxylate salt according to the present invention is within the above range.

Illustrative examples of the carboxylic acid having 8 to 10 carbon atoms include octanoic acid, 2-ethylhexanoic acid, isooctanoic acid, nonanoic acid, isononanoic acid, neononanoic acid, decanoic acid, isodecanoic acid, and neodecanoic acid. These carboxylic acids are used singly or in a combination of two or more of them so as to achieve a predetermined average branching degree. It is desirable to use a combination of two or more carboxylic acids, in which 2-ethylhexanoic acid, isononanoic acid, or neodecanoic acid is included as the main component therein.

In the present invention, the ratio of the carboxylic acids having 8 to 10 carbon atoms in the whole of the carboxylic acids that form the zinc carboxylate salt according to the present invention is measured by a gas chromatography, a liquid chromatography-mass spectrometry, or other methods with regard to the types and amounts of the carboxylic acids; then, on the basis of these measurement results the ratio is calculated. For example, when analyzing the raw material carboxylic acid for the zinc carboxylate salt according to the present invention (in the case that the carboxylic acid that forms the raw material zinc carboxylate salt is composed of two or more carboxylic acids, a mixture of these carboxylic acids), that is, when analyzing the carboxylic acid before reacting with a zine compound to form the zine carboxylate salt according to the present invention (in the case that the carboxylic acid that forms the zine carboxylate salt according to the present invention is composed of two or more carboxylic acids, a mixture of those carboxylic acids) by a gas chromatography, a portion of the carboxylic acid is collected and methyl-esterified, and then, this is introduced into a gas chromatography instrument, heated to 350° C. or higher, and passed through a column with a carrier gas to identify and determine the type and amount of the carboxylic acid from the retention time and peak area of the signal, these being obtained by a detector; and from the results, the ratio of the carboxylic acids having 8 to 10 carbon atoms in the whole of the carboxylic acids that form the zinc carboxylate salt according to the present invention can be calculated. Also, for example, a portion of the raw material zinc carboxylate salt before being added to the core particle dispersion solution is collected, and a strong acid such as hydrochloric acid or nitric acid is added to separate the carboxylic acids, followed by methyl-esterification, and then this is introduced into a sample evaporation chamber of a gas chromatography instrument, heated to 350° C. or higher, and passed through a column with a carrier gas. Then, the types and amounts of the carboxylic acids are identified and determined from the retention times and peak areas of the signals obtained by the detector; and from the results, the ratio of the carboxylic acids having 8 to 10 carbon atoms in the whole of the carboxylic acids that form the zinc carboxylate salt according to the present invention can be calculated.

The average branching degree of the whole of the carboxylic acids that form the zinc carboxylate salt according to the present invention is in the range of 1.1 to 2.9, preferably in the range of 1.3 to 2.7, and especially preferably in the range of 1.5 to 2.5. In other words, upon measuring the whole of the carboxylic acids that form the zinc carboxylate salt according to the present invention, the average branching degree is in the range of 1.1 to 2.9, preferably in the range of 1.3 to 2.7, and especially preferably in the range of 1.5 to 2.5. When the average branching degree of the whole of the carboxylic acids that form the zinc carboxylate salt is within the above range, a high solubility in an organic hydrocarbon solvent and an improved workability can be realized. In addition, the semiconductor nanoparticle having excellent optical properties can be obtained by using the zine carboxylate salt according to the present invention as the Zn precursor during production of the semiconductor nanoparticle. Probably, the reason for this is because when the average branching degree is within the above range, the zinc carboxylate salt has a certain bulkiness, thereby reacting with other shell-forming precursors on the surface of the core particle, resulting in obtaining the semiconductor nanoparticle having excellent optical properties.

In the present invention, the average branching degree of the whole of the carboxylic acids that form the zinc carboxylate salt according to the present invention means the branching degree of the alkyl group from the main chain of these carboxylic acids. First, the measurement sample is analyzed by a gas chromatography or other means, and then the average molecular weight of the carboxylic acids (14n+32) is calculated from the composition ratio obtained. The measurement sample is analyzed by ¹H-NMR, and from the NMR chart obtained, the integral of the chemical shift indicating the hydrogen of all the alkyl chains of the carboxylic acid is taken as 2n−1, and the integral of the chemical shift δ in the range of 0.7 to 1.1 ppm indicating the hydrogen of the primary carbon is divided by 3 to obtain the number of methyl groups in that carboxylic acid. The branching degree is calculated by subtracting 1, the number of terminal methyl groups in the main chain structure, from the number of methyl groups obtained. For example, a portion of the raw material carboxylic acid (in the case that the carboxylic acid that forms the zine carboxylate salt according to the present invention is composed of two or more carboxylic acids, a mixture of those carboxylic acids) for making the zine carboxylate salt according to the present invention, that is, the carboxylic acid (in the case that the carboxylic acid that forms the raw material zine carboxylate salt is composed of two or more carboxylic acids, a mixture of those carboxylic acids) before reacting with a zine compound to form the raw material zinc carboxylate salt, is collected and analyzed by ¹H-NMR; then the calculation is made by subtracting 1, the number of terminal methyl groups in the main chain structure, from the number of methyl groups obtained from the obtained NMR chart. For example, a portion of the zinc carboxylate salt according to the present invention before adding to the core particle dispersion solution is taken and added with a strong acid such as hydrochloric acid or nitric acid to separate the carboxylic acid for producing the raw material; then, this is analyzed by ¹H-NMR, and the calculation is made by subtracting 1, the number of terminal methyl groups in the main chain structure, from the number of methyl groups obtained from the obtained NMR chart.

The zinc carboxylate salt according to the present invention is obtained by the reaction of a carboxylic acid with a zinc raw material. The zinc carboxylate salt is generally formed by the method called a direct method or a double decomposition method. In the present invention, either method may be used. The direct method is the method in which a molten carboxylic acid is caused to directly react with a metal oxide or a metal hydroxide to obtain a metal carboxylate salt. On the other hand, the double decomposition method is to obtain a metal carboxylate salt by reacting an aqueous solution of an alkali metal salt of a carboxylic acid with an inorganic metal salt. The zinc carboxylate salt according to the present invention may be formed by any of the direct method and the double decomposition method; but in view of being contaminated with a less amount of water into the zinc carboxylate salt, the zinc carboxylate salt formed by the direct method is more preferable.

When preparing the zinc salt with two or more carboxylic acids, the salt may be prepared after previously mixing the carboxylic acids having known degrees of branching so as to adjust the branching degree before preparation, or the salt may be prepared by mixing the salts after preparation of the zine carboxylate so as to adjust the branching degree. From the production viewpoint, mixing the carboxylic acids in advance is preferable. The zinc carboxylate salt may also be produced in a solvent that is used to produce the semiconductor nanoparticle.

In view of excellent dispersibility and solubility stability in a solvent, the zinc carboxylate salt according to the present invention has the rate of change in viscosity, indicated by the following formula (1), preferably in the range of 95.0% to 100.0%, more preferably in the range of 97.0% to 100.0%, and especially preferably in the range of 97.0% to 99.9%.

$$\begin{matrix}General formula ( 1 ) & \\ Rate of change in viscosity ( \% ) = ( ( viscosity ( Pa * s ) at 130 °C - viscosity ( Pa * s ) at 50 °C ) / viscosity ( Pa * s ) at 50 °C ) \times 100 & ( 1 )\end{matrix}$$

(In the formula, the viscosity (Pa*s) at 130° C. is the value obtained by measuring a zinc carboxylate salt at a temperature of 130° C. by a dynamic viscoelasticity analyzer, and the viscosity at 50° C. is the value obtained by measuring a zinc carboxylate salt at a temperature of 50° C. by a dynamic viscoelasticity analyzer.)

The group VI element precursor to be added to the core particle dispersion solution at the shell-forming process (1), namely, the group VI element precursor to be reacted with the zine precursor, is a Se precursor, a S precursor, or a Te precursor. The group VI element precursor may be one kind alone or a combination of two or more kinds; but it is preferable to include at least a Se precursor. In other words, the group VI element precursor to be reacted with the zinc precursor may be a precursor of any one of the VI elements, such as a Se precursor alone, or a combination of two or more VI elements, such as a Se precursor and a S precursor, a Se precursor and a Te precursor, a Se precursor, a S precursor, and a Te precursor, among other combinations.

At the shell-forming process (1), there is no particular restriction on the Se precursor. Illustrative examples thereof include a trialkylphosphine selenide and selenol. A trialkylphosphine selenide is preferable as the Se precursor. The Se precursor may be one kind alone or a combination of two or more kinds.

At the shell-forming process (1), there is no particular restriction on the S precursor. Illustrative examples thereof include: trialkylphosphine sulfides such as trioctylphosphine sulfide and tributylphosphine sulfide; a thiol; and bis(trimethylsilyl) sulfide. Trioctylphosphine sulfide is preferable as the S precursor. The S precursor may be one kind alone or a combination of two or more kinds.

At the shell-forming process (1), there is no particular restriction on the Te precursor. Illustrative examples thereof include trioctylphosphine telluride. Trioctylphosphine telluride is preferable as the Te precursor. The Te precursor may be one kind alone or a combination of two or more kinds.

At the shell-forming process (1), when only the Se precursor is used as the Group VI element precursor, a shell layer including zinc and Se is formed, and when Se and S precursors are used together, a shell layer including zinc, Se, and S is formed. When Se and Te precursors are used together, a shell layer including zinc, Se, and Te is formed. When Se, S, and Te precursors are used together, a shell layer including zinc, Se, S, and Te is formed.

There is no particular restriction on the method in which at the shell-forming process (1), the raw material zinc carboxylate salt and the group VI element precursor are added to the core particle dispersion solution to form the shell that includes zinc and the group VI element on the surface of the core particle by reacting the raw material zinc carboxylate salt with the group VI element precursor in the presence of the core particle. Any method may be used as long as both the raw material zinc carboxylate salt and the group VI element precursor are added to the core particle dispersion solution, thereby causing the reaction of the raw material zinc carboxylate salt with the group VI element precursor in the presence of the core particle.

At the shell-forming process (1), the raw material zine carboxylate salt and the group VI element precursor are added to the core particle dispersion solution, which is then followed by the reaction between the raw material zinc carboxylate salt and the group VI element precursor in the presence of the core particle to form a shell that includes zinc and a group VI element on the surface of the core particle.

When the raw material zinc carboxylate salt and the group VI element precursor are added to the core particle dispersion solution at the shell-forming process (1), the temperature of the core particle dispersion solution is chosen as appropriate from the range of 180 to 320° C. When the temperature of the core particle dispersion solution upon reacting the raw material zine carboxylate salt with the group VI element precursor is within the above range, the added raw material zinc carboxylate salt and the group VI element precursor can form on the core particle the shell that is unlikely to cause the non-emission recombination of excitons through the defect level; thus, the core/shell type semiconductor nanoparticle having excellent optical properties can be obtained.

When adding the raw material zinc carboxylate salt into the core particle dispersion solution at the shell-forming process (1), the addition time is chosen as appropriate from the range of 5 to 600 minutes. When the addition time of the solution of the raw material zinc carboxylate salt or the raw material zinc carboxylate salt and the solution of the group VI element precursor to the core particle dispersion solution is within the above range, the added shell precursors can efficiently form the shell on the surface of the core particle.

The addition time for adding the group VI element precursor to the core particle dispersion at the shell-forming process (1) is appropriately chosen from the range of 5 to 600 minutes. When the addition time of the solution of the group VI element precursor or the solution of the group VI element precursor to the core particle dispersion solution is within the above range, the added shell precursor can efficiently form the shell on the surface of the core particle.

At the shell-forming process (1), the reaction between the raw material zine carboxylate salt and the group VI element precursor can be conducted in the presence of a dispersant. Illustrative examples of the dispersant to be present in the core particle dispersion solution include: amines such as oleylamine and trioctylamine; carboxylic acids such as oleic acid; and thiols such as dodecanethiol. The amount of the dispersant used is chosen as appropriate. The amount thereof in terms of the mole ratio to In in the core particle is preferably in the range of 5 to 200, and the amount in terms of the mole ratio to In in the core particle is more preferably in the range of 10 to 100.

At the shell-forming process (1), the reaction of the raw material zinc carboxylate salt with the group VI element precursor may be conducted in the presence of a halogen precursor. There is no particular restriction on the halogen precursor. Illustrative examples thereof include HF, HCl, HBr, and HI; carboxylic acid halides such oleyl chloride, oleyl bromide, octanoyl chloride, and octanoyl bromide; and metal halides such as zinc chloride, indium chloride, and gallium chloride. The amount of the halogen used is chosen as appropriate; the amount in terms of the mole ratio to In in the core particle is preferably in the range of 0.3 to 100.0, and the amount in terms of the mole ratio to In in the core particle is more preferably in the range of 0.3 to 30.0. At the shell-forming process (1), when the halogen precursor is made present in the core particle dispersion solution, the core/shell type semiconductor nanoparticle having the halogen on the surface of the core particle or in the shell layer thereof can be obtained.

At the shell-forming process (1), the reaction between the raw material zine carboxylate salt and the group VI element precursor may be conducted in the presence of, in addition to the above substances, a carboxylic acid, an amine, a thiol, a phosphine, a phosphine oxide, a phosphine, a phosphonic acid, or the like, as needed.

In the method for producing the semiconductor nanoparticle using the zinc carboxylate salt according to the present invention, the core particle obtained after synthesis of the core particle may be used as the core particle for the shell-forming process (1) without being purified. Namely, the core particle that has not gone through a purification process may be used as the core particle for the shell-forming process (1). In other words, the reaction solution in which the core particles are dispersed after the synthesis of the core particles may be used as the core particle dispersion solution for the shell-forming process (1). At the shell-forming process (1), the zinc salt of the carboxylic acids having a specific carbon number and a specific branching degree is used as the zinc precursor to form the shell layer. The inventors of the present invention presume that the zine carboxylate salt and the group VI element precursor can readily react on the surface of the core particle, making it difficult for impurities in the dispersing medium to be incorporated into the shell layer at the time of formation of the shell and making it possible to form the shell having the defect level suppressed on the core surface. Therefore, the core particle that has not gone through the purification process can be used as the core particle for the shell-forming process (1).

The method for producing the semiconductor nanoparticle using the zinc carboxylate salt according to the present invention is simple, because in order to form the shell layer at the shell-forming process (1), what is required is only one-time addition of all the quantities of the raw material zinc carboxylate salt and the group VI element precursor to be used to form the shell into the core particle dispersion solution. In the method for producing the core/shell type semiconductor nanoparticle according to the present invention, the raw material zine carboxylate, which is the zine salt of the carboxylic acids having a specific carbon number and a specific branching degree, is used as the group II element precursor to be added to the core particle dispersion solution so as to be reacted with the group VI element precursor on the surface of the core particle. With this, the core/shell type semiconductor nanoparticle having excellent optical properties can be obtained even if the group II element precursor and the group VI element precursor are not brought into contact with the core particle separately and alternately for multiple times, in other words, even if all the group II element precursor and the group VI element precursor are brought into contact and reacted with the core particle at one time. In particular, the method for producing the core/shell type semiconductor nanoparticle according to the present invention is highly effective to obtain the core/shell type semiconductor nanoparticle having excellent optical properties, even if all the quantities of the group II element precursor and the group VI element precursor are brought into contact and reacted with the core particle at a time at the shell-forming process (1).

In the zinc carboxylate salt according to the present invention, the ratio of the carboxylic acids having 8 to 10 carbon atoms in the whole of the carboxylic acids that form the zinc carboxylate salt is made 80.0% by mass or more, preferably 85.0% by mass or more, more preferably 90.0% by mass or more, and especially preferably 100.0% by mass, and the average branching degree of the whole of the carboxylic acids that form the raw material zinc carboxylate salt is made in the range of 1.1 to 2.9, preferably in the range of 1.3 to 2.7, and especially preferably in the range of 1.5 to 2.5. By using this zinc carboxylate salt at the shell-forming process (1), the core/shell type semiconductor nanoparticle having a narrow full width at half maximum (FWHM) and a high quantum yield (QY) can be obtained.

In general, in the production of the core/shell type semiconductor nanoparticle, the core/shell type semiconductor nanoparticle for red emission with the emission peak wavelength λₘₐₓ of 590 to 650 nm has a larger particle size than that of the core/shell type semiconductor nanoparticle for green emission with the emission peak wavelength λₘₐₓ of 500 to 560 nm; thus, it is difficult to obtain the core/shell type semiconductor nanoparticle having a narrow full width at half maximum (FWHM) and a high quantum yield (QY).

In the method for producing the semiconductor nanoparticle using the zinc carboxylate salt according to the present invention, at the shell forming process (1), the group VI element precursor and the raw material zinc carboxylate salt are added to the core particle dispersion solution to cause the raw material zinc carboxylate salt to react with the group VI element precursor. In addition, the ratio of the carboxylic acids having 8 to 10 carbon atoms in the whole of the carboxylic acids that form the raw material zinc carboxylate salt is made 80.0% by mass or more, preferably 85.0% by mass or more, more preferably 90.0% by mass or more, and especially preferably 100.0% by mass, and the average branching degree of the whole of the carboxylic acids that form the raw material zinc carboxylate salt is made in the range of 1.1 to 2.9, preferably in the range of 1.3 to 2.7, and especially preferably in the range of 1.5 to 2.5. In other words, by using the zinc carboxylate salt according to the present invention, the core/shell type semiconductor nanoparticle having a narrow full width at half maximum (FWHM) and a high quantum yield (QY) can be obtained also in the production of the core/shell type semiconductor nanoparticle for red emission with the emission peak wavelength Amax of 590 to 650 nm. Although the mechanism for this is not yet clear, the inventors of the present invention presume that the zinc salt of the carboxylic acids having a specific carbon number and a specific branching degree is readily present on the surface of the core particle of the semiconductor nanoparticle, and whereby the reaction of the zine salt of the carboxylic acids having a specific carbon number and a specific branching degree present on the surface of the core particle with the group VI element precursor results in formation of the II-VI group shell having generation of the defect level on the surface of the core particle suppressed.

In the method for producing the semiconductor nanoparticle using the zinc carboxylate salt according to the present invention, after conducting the shell-forming process (1), the generated particle having the core/shell type structure may be obtained as the target product of the core/shell type semiconductor particle, or, furthermore, using the generated core/shell type particle, the core/shell type semiconductor nanoparticle having two or more shell layers may be obtained by conducting the shell-forming process one or two or more times. In other words, the method for producing the semiconductor nanoparticle using the zinc carboxylate salt according to the present invention may have, in addition to the shell-forming process (1), one or two or more shell-forming processes to form the shell on the core/shell type particle that includes zine and a group VI element obtained by conducting the shell-forming process (1). The shell-forming process, which is conducted one or two or more times, is the same as the shell-forming process (1) described above. In other words, the shell-forming process may be conducted in the same way as the shell-forming process (1), except that the core/shell type particle having one or more shell layers formed on the surface of the core particle is used in place of the core particle. The shell-forming process that is conducted one or two or more times may be a method other than the method similar to the above shell-forming process (1).

As for the method for producing the core/shell type semiconductor nanoparticle having two shell layers formed on the surface of the core particle, a method for producing the core/shell type semiconductor nanoparticle may be mentioned, in which the method has the shell-forming process (1) and a shell-forming process (2) at which the raw material zinc carboxylate salt and the group VI element precursor are added to the dispersion solution of “particles composed of the core particle and one shell layer formed on the surface of the core particle” obtained by conducting the shell-forming process (1) to form a shell that includes zinc and the group VI element on the surface of the “particles composed of the core particle and one shell layer formed on the surface of the core particle.”

In the method for producing the semiconductor nanoparticle using the zinc carboxylate salt according to the present invention, in the case where the shell-forming process is conducted one or two or more times after the shell-forming process (1) above is conducted, at the shell-forming process (1), the carboxylic acid having a branched chain derived from the raw material zinc carboxylate salt used as the zinc precursor is coordinated to the surface of the core/shell type semiconductor nanoparticle obtained by the method for production of the semiconductor nanoparticle using the zinc carboxylate salt according to the present invention. The carboxylic acid having the branched chain that is coordinated to the surface of the core/shell type semiconductor nanoparticle acts as the ligand that enhances the dispersibility of the core/shell type semiconductor nanoparticle into a dispersing medium.

In addition, as for the method for producing the core/shell type semiconductor nanoparticle having (n+1) shell layers formed on the surface of the core particle, a method for producing the core/shell type semiconductor nanoparticle may be mentioned, in which the method has the shell-forming process (1) and a process at which a shell-forming process (x) is repeated n times at which the raw material zinc carboxylate salt and the group VI element precursor are added to the dispersion solution of “particles composed of the core particle and one or more shell layers formed on the surface of the core particle” obtained by conducting the shell-forming process above to form the shell that includes zinc and the group VI element on the surface of the “particles composed of the core particle and one or more shell layers formed on the surface of the core particle.”

In the method for producing the semiconductor nanoparticle using the zinc carboxylate salt according to the present invention, in the case where the shell-forming process (x) is repeatedly conducted one or two or more times after the shell-forming process (1) above is conducted, in the shell-forming process (1) and the shell-forming process (x), the carboxylic acid derived from the raw material zinc carboxylate salt used as the zinc precursor is coordinated to the surface of the core/shell type semiconductor nanoparticle obtained by the method for producing the semiconductor nanoparticle using the zinc carboxylate salt according to the present invention. The carboxylic acid that is coordinated to the surface of the core/shell type semiconductor nanoparticle acts as the ligand that enhances the dispersibility of the core/shell type semiconductor nanoparticle into the dispersion solution.

### <Measurement>

Elemental analysis of the semiconductor nanoparticle may be conducted using a high-frequency inductively coupled plasma emission spectrometer (ICP) or an X-ray fluorescence spectrometer (XRF). In the ICP measurement, purified semiconductor nanoparticles are dissolved in nitric acid, heated, diluted with water, and measured by means of the calibration curve method using an ICP emission spectrometer (ICPS-8100; manufactured by Shimadzu Corp.). In the XRF measurement, a sample having the dispersion solution impregnated in a filter paper is placed in a sampling holder to carry out the quantitative analysis using an X-ray fluorescence spectrometer (ZSX100e; manufactured by Rigaku Corp.).

The optical identification of the semiconductor nanoparticle may be carried out by measuring with a fluorescence quantum yield measurement system (QE-2100; manufactured by Otsuka Electronics Co., Ltd.) and a visible-ultraviolet spectrophotometer (V670; manufactured by JASCO Corp.). The emission spectrum is obtained by irradiating an excitation light to a dispersion solution of the semiconductor nanoparticles in a dispersing medium. The peak wavelength (Xmax), the fluorescence quantum yield (QY), and the full width at half maximum (FWHM) are calculated from the re-excitation-corrected emission spectrum obtained by excluding the re-excited fluorescence emission spectrum that is fluorescence-emitted by re-excitation from the fluorescence spectrum obtained as described above. Illustrative examples of the dispersing medium include normal hexane, octadecene, toluene, acetone, and PGMEA. The excitation light used for the measurement is a single beam of 450 nm, and the dispersion solution is made of the semiconductor nanoparticle whose concentration is adjusted so as to give the absorbance of 20 to 30%. On the other hand, the absorption spectrum may be measured by irradiating UV to visible light to a dispersion solution of the semiconductor nanoparticles in a dispersing medium.

With regard to the ligand that is coordinated to the core/shell type semiconductor nanoparticle, identification of its type and calculation of its mole fraction may be done using a gas chromatography. The core/shell type semiconductor nanoparticles are introduced into a sample vaporization chamber, heated at 350° C. or higher, and passed through a column with a carrier gas; with this, the type and amount of each ligand are identified and calculated from the retention time and peak area of the signal, obtained with a detector. From the obtained type and amount of each ligand, the existing type and ratio of the ligand that is coordinated to the core/shell type semiconductor nanoparticle may be identified and calculated.

The composition, method, procedure, process, and the like described in this specification are examples and thus, do not limit the present invention, so that many variations are applicable within the scope of the present invention.

The zinc salt according to the present invention, used in the production of the semiconductor nanoparticle is a zine carboxylate salt in which
a ratio of carboxylic acids having 8 to 10 carbon atoms in whole of carboxylic acids that form the zine carboxylate salt is 80.0% by mass or more, and
an average branching degree of the whole of the carboxylic acids that form the zinc carboxylate salt is in the range of 1.1 to 2.9.

The zinc salt according to the present invention, used in the production of the semiconductor nanoparticle is used to form a shell in the production of the core/shell type semiconductor nanoparticle. The zine salt according to the present invention, used in the production of the semiconductor nanoparticle is a zinc carboxylate salt, in which the ratio of carboxylic acids having 8 to 10 carbon atoms in the whole of the carboxylic acids that form the zinc carboxylate salt is 80.0% by mass or more, preferably 85.0% by mass or more, more preferably 90.0% by mass or more, and especially preferably 100.0% by mass, and the average branching degree of the whole of the carboxylic acids that form the zinc carboxylate salt is in the range of 1.1 to 2.9, preferably in the range of 1.3 to 2.7, and especially preferably in the range of 1.5 to 2.5.

Hereinafter, the present invention will be described based on specific experimental examples, but the present invention is not limited to these examples.

### EXAMPLES

### <Production of Zinc Carboxylate>

The zinc salt with branched carboxylic acids was prepared according to the following method. The carboxylic acids used were 3,5,5-trimethylhexanoic acid (reagent with >98.0% purity; manufactured by Tokyo Chemical Industry Co., Ltd.), neodecanoic acid (reagent manufactured by FUJIFILM Wako Pure Chemical Corp.), 2-ethylhexanoic acid (reagent with >99.0% purity; manufactured by Tokyo Chemical Industry Co., Ltd.), and decanoic acid (NAA-102; manufactured by NOF Corp.). The analysis results of the carbon number compositions of carboxylic acids are summarized in Table 1 and the blending ratios of the carboxylic acids are summarized in Table 2.

### Example 1

Neodecanoic acid (158 g, 0.91 mol), 2-ethylhexanoic acid (91.8 g, 0.64 mol), and decanoic acid (47.1 g, 0.27 mol) were heated at 40° C. with stirring to prepare a carboxylic acid mixture. The prepared carboxylic acid mixture and zinc oxide (58.8 g, 0.90 mol) were charged into a separable flask equipped with a water quantifying receiver; and then, they were stirred and heated to 170° C. under a nitrogen atmosphere. The temperature was kept at 170° C. for 2 hours after removing the generated water through the water quantifying receiver. After evacuation for 1 hour, purging with nitrogen was performed, which was then followed by cooling to room temperature (25° C.) to obtain zinc carboxylate salt 1.

### Example 2

A carboxylic acid mixture was prepared by stirring 3,5,5-trimethylhexanoic acid (144 g, 0.91 mol) and neodecanoic acid (158 g, 0.91 mol) with heating at 40° C. The prepared carboxylic acid mixture and zinc oxide (58.8 g, 0.90 mol) were charged into a separable flask equipped with a water quantifying receiver; and then, they were stirred and heated to 170° C. under a nitrogen atmosphere. The temperature was kept at 170° C. for 2 hours after removing the generated water through the water quantifying receiver. After evacuation for 1 hour, purging with nitrogen was performed, which was then followed by cooling to room temperature (25° C.) to obtain zinc carboxylate salt 2.

### Example 3

A carboxylic acid mixture was prepared by stirring 3,5,5-trimethylhexanoic acid (71.9 g, 0.45 mol) and neodecanoic acid (237 g, 1.36 mol) with heating at 40° C. The prepared carboxylic acid mixture and zinc oxide (58.8 g, 0.90 mol) were charged into a separable flask equipped with a water quantifying receiver; and then, they were stirred and heated to 170° C. under a nitrogen atmosphere. The temperature was kept at 170° C. for 2 hours after removing the generated water through the water quantifying receiver. After evacuation for 1 hour, purging with nitrogen was performed, which was then followed by cooling to room temperature (25° C.) to obtain zine carboxylate salt 3.

### Example 4

Neodecanoic acid (316 g, 1.82 mol) and zinc oxide (58.8 g, 0.90 mol) were charged into a separable flask equipped with a water quantifying receiver; and then, they were stirred and heated to 170° C. under a nitrogen atmosphere. The temperature was kept at 170° C. for 2 hours after removing the generated water through the water quantifying receiver. After evacuation for 1 hour, purging with nitrogen was performed and cooled to room temperature (25° C.) to obtain zinc carboxylate salt 4.

### Example 5

Neodecanoic acid (261 g, 1.50 mol) and decanoic acid (54.6 g, 0.32 mol) were stirred with heating at 40° C. to prepare the carboxylic acid mixture. The prepared carboxylic acid mixture and zinc oxide (58.8 g, 0.90 mol) were charged into a separable flask equipped with a water quantifying receiver; and then, they were stirred and heated to 170° C. under a nitrogen atmosphere. The temperature was kept at 170° C. for 2 hours after removing the generated water through the water quantifying receiver. After evacuation for 1 hour, purging with nitrogen was performed and cooled to room temperature (25° C.) to obtain zine carboxylate salt 5.

### Example 6

A carboxylic acid mixture was prepared by stirring 3,5,5-trimethylhexanoic acid (95.8 g, 0.61 mol), 2-ethylhexanoic acid (17.8 g, 0.12 mol), and decanoic acid (188 g, 1.09 mol) with heating at 40° C. The prepared carboxylic acid mixture and zinc oxide (58.8 g, 0.90 mol) were charged into a separable flask equipped with a water quantifying receiver; and then, they were stirred and heated to 170° C. under a nitrogen atmosphere. The temperature was kept at 170° C. for 2 hours after removing the generated water through the water quantifying receiver. After evacuation for 1 hour, purging with nitrogen was performed and cooled to room temperature (25° C.) to obtain zinc carboxylate salt 6.

### Comparative Example 1

3,5,5-Trimethylhexanoic acid (288 g, 1.82 mol) and zinc oxide (58.8 g, 0.90 mol) were charged into a separable flask equipped with a water quantifying receiver; and then, they were stirred and heated to 170° C. under a nitrogen atmosphere. The temperature was kept at 170° C. for 2 hours after removing the generated water through the water quantifying receiver. After evacuation for 1 hour, purging with nitrogen was performed and cooled to room temperature (25° C.) to obtain zine carboxylate salt 7.

### Comparative Example 2

Decanoic acid (316 g, 1.82 mol) and water (2000 g) were charged into a separable flask and heated to 60° C. Next, an aqueous 48.0 wt % sodium hydroxide solution (154 g, 1.82 mol) was added, and then, the resulting mixture was stirred for 20 minutes, followed by dropwise addition of an aqueous solution of 25.0 wt % zinc sulfate (650 g, 2.00 mol) over 60 minutes. After the dropwise addition was completed, the resulting zinc carboxylate slurry was suction-filtrated and washed three times with 1000 g of water. The resulting cake was allowed to be placed in a shelf dryer at 60° C. for 36 hours, and then cooled to room temperature (25° C.) to obtain zine carboxylate salt 8.

### <Measurement of Zinc Carboxylate Salt>

The physical properties of the zinc carboxylate salts 1 to 8 obtained above were measured. The results are summarized in Table 3.

### (Ratio of C8 to C10 Carboxylic Acids)

The ratios of the C8 to C10 carboxylic acids (% by mass) in the zinc carboxylate salts 1 to 8 were calculated from the carbon number compositions of the carboxylic acids in Table 1 and the blending ratio of the carboxylic acids in Table 2.

### (Average Branching Degree)

The average branching degree of the zine carboxylate salts 1 to 8 was calculated from the branching degrees of the carboxylic acids in Table 1 and the blending ratios of the carboxylic acids in Table 2.

### (Metal Content: Zn Content)

Each of the zinc carboxylate salts 1 to 8 was weighed (0.1 g) and heated in a porcelain crucible at 650° C. for 4 hours to remove organic matters. Hydrochloric acid (1 ml) was added to the residue to dissolve it, and water was added to make it 100 ml. The metal content (Zn content) was measured by an atomic absorption spectrophotometry using this solution as the measurement sample.

### (Viscosity)

The dynamic viscosity of each of the zinc carboxylate salts 1 to 8 was measured using a dynamic viscoelasticity analyzer (Modular Concept Rheometer MCR302; manufactured by Anton Paar GmbH). The zinc carboxylate was placed on a rheometer hot plate and heated to 130° C., and the temperature thereof was kept with a sample cover. The dynamic viscosity was measured when the rotation speed was swept from 1.0 rpm to 1000 rpm using a cone plate (CP25-2); and the viscosity (Pa*s) at 130° C. was calculated at a rotation speed of 150 rpm. The temperature was then lowered to 50° C., and the dynamic viscosity was measured when the rotation speed was swept from 1.0 rpm to 1000 rpm in the same manner; and the viscosity (Pass) at 50° C. was calculated at a rotation speed of 150 rpm.

### (Rate of Change in Viscosity)

The rate of change in viscosity with respect to the temperature change was calculated by taking the difference between the viscosity (Pa*s) at 130° C. and the viscosity (Pa*s) at 50° C., followed by dividing the difference with the viscosity (Pats) at 50° C. and then multiplying the obtained value by 100.

$$Rate of change in viscosity ( \% ) = ( ( viscosity ( Pa * s ) at 130 °C - viscosity ( Pa * s ) at 50 °C ) / viscosity ( Pa * s ) at 50 °C ) \times 100$$

Semiconductor nanoparticles and semiconductor nanoparticle composites were prepared according to the following method, and the optical properties of the resulting semiconductor nanoparticle and semiconductor nanoparticle composites were measured.

### <Synthesis of Core Particle>

Indium acetate (0.5 mmol), myristic acid (1.5 mmol), zinc myristate (0.2 mmol), and octadecene (10 mL) were charged into a two-necked flask, then the flask was evacuated and heated to 120° C. under vacuum (<10 Pa). After holding for 30 minutes since when the degree of vacuum reached lower than 10 Pa, nitrogen was introduced into the flask and the flask was cooled to room temperature (25° C.) to obtain an In precursor.

Tris(trimethylsilyl)phosphine was mixed with tri-u-octylphosphine so as to give a molar concentration of 0.2 M under a nitrogen atmosphere in a glove box to obtain a P precursor.

Next, 2 mL of the P precursor was charged into the In precursor at room temperature (25° C.) under a nitrogen atmosphere, and then the temperature thereof was raised to 300° C. at a rate of 30° C./min. After holding at 300° C. for 2 min, the reaction solution was cooled to room temperature to obtain the reaction solution as a dispersion solution of InP core particles.

### <Solution of Zinc Precursor>

The solution of each zinc precursor was obtained by mixing the zine carboxylate salt listed in Table 2 with octadecene so as to give a zinc molar concentration of 0.3 M; then, this was evacuated at 100° C. for 1 hour, replaced with nitrogen, and then cooled to room temperature (25° C.).

### <Se Precursor Solution>

A solution of a Se precursor was obtained by mixing 100 mmol of powdered selenium and 50 mL of tri-n-octylphosphine under a nitrogen atmosphere with stirring until the selenium powder was completely dissolved.

### <S Precursor Solution>

A solution of an S precursor was obtained by mixing 100 mmol of powdered sulfur and 50 mL of tri-n-octylphosphine under a nitrogen atmosphere with stirring until the sulfur powder was completely dissolved.

### <Production of Core/Shell Type Semiconductor Nanoparticle>

### Example 7

To 10 mL of the dispersion solution of InP core particles (In: 0.4 mmol), 5 mL of trioctylamine was added; then, the temperature of the dispersion solution of the InP core particles was raised to 230° C. When the temperature of the dispersion solution of the InP core particles reached 230° C., 20 mL of the solution of the zine precursor and 2.0 mL of the solution of the Se precursor described in Table 4 were added within 1 minute; then, the temperature of the dispersion solution of the InP core particles was raised to 300° C. at a rate of 1° C./minute. Then, 180 minutes after the temperature of the dispersion solution of the InP core particles reached 300° C., the heating was stopped, which was then followed by cooling to room temperature (25° C.) to obtain the dispersion solution of the core/shell type semiconductor nanoparticles (reaction solution).

Next, acetone was added to the resulting dispersion solution of the core/shell type semiconductor nanoparticles to agglomerate the semiconductor nanoparticles. Then, after centrifugation (4000 rpm, 10 min), the supernatant was removed, and the core/shell type semiconductor nanoparticles were redispersed in hexane. This was repeated to obtain purified core/shell type semiconductor nanoparticles.

The optical properties of the resulting core/shell type semiconductor nanoparticle were measured. The results are summarized in Table 5.

In the measurement of optical properties of the semiconductor nanoparticle, the excitation wavelength was a single wavelength of 450 nm. The same is true for the measurement of the optical properties of the following semiconductor nanoparticles.

### <Production of Core/Shell/Shell Semiconductor Nanoparticle>

### Examples 8 to 13 and Comparative Examples 3 and 4

The same procedure as in each of the above Examples or Comparative Examples was performed to obtain the dispersion solution of the core/shell semiconductor nanoparticles (reaction solution). The resulting dispersion solution of the core/shell semiconductor nanoparticles (reaction solution) was then heated to 300° C. After the temperature reached 300° C., the addition of the solution of the zinc precursor described in Table 4 to the dispersion solution of the core/shell semiconductor nanoparticles (reaction solution) was started at a rate of 0.2 mL/min with simultaneous addition of the solution of the Se precursor and the solution of the S precursor both at a rate of 0.03 mL/min. One hundred minutes after the start of the addition of the solution of the zinc precursor and the solution of the S precursor, the addition of both the solutions were terminated simultaneously (addition time: 100 min). Then, 180 minutes after the addition was over, the heating was stopped, which was then followed by cooling to room temperature (25° C.) to obtain the core/shell/shell semiconductor nanoparticles. In this way, the dispersion solution thereof (reaction solution) was obtained.

Next, acetone was added to the resulting dispersion solution of the core/shell/shell semiconductor nanoparticles to agglomerate the semiconductor nanoparticles. Then, after centrifugation (4000 rpm, 10 min), the supernatant was removed and the core/shell/shell semiconductor nanoparticles were redispersed in hexane. This was repeated to obtain purified core/shell/shell semiconductor nanoparticles.

The optical properties of the resulting core/shell/shell semiconductor nanoparticle were measured. The results are summarized in Table 5.

TABLE 1

| | | | | | C7 or | |
|---|---|---|---|---|---|---|
| | | | | | less, | |
| | | C8 | C9 | C10 | or C11 | Average |
| Carboxylic acid: reagent/product | | (% by | (% by | (% by | (% by | branching |
| name | Symbol | mass) | mass) | mass) | mass) | degree |
| 3,5,5-Trimethylhexanoic acid | A | 0.6 | 98.5 | 0.5 | 0.4 | 3.1 |
| Neodecanoic acid | B | 0.0 | 0.6 | 90.9 | 8.5 | 2.3 |
| 2-Ethylhexanoic acid | C | 99.5 | 0.0 | 0.3 | 0.2 | 1.0 |
| Decanoic acid (NAA-102) | D | 0.2 | 0.0 | 99.5 | 0.3 | 0.0 |

TABLE 2

| | | Blending ratio of carboxylic | | | |
|---|---|---|---|---|---|
| | | acids (% by mass) | | | |
| | Blended carboxylic acid | A | B | C | D |
| Example 1 | Zinc carboxylate salt 1 | 0.0 | 50.0 | 35.0 | 15.0 |
| Example 2 | Zinc carboxylate salt 2 | 50.0 | 50.0 | 0.0 | 0.0 |
| Example 3 | Zinc carboxylate salt 3 | 25.0 | 75.0 | 0.0 | 0.0 |
| Example 4 | Zinc carboxylate salt 4 | 0.0 | 100.0 | 0.0 | 0.0 |
| Example 5 | Zinc carboxylate salt 5 | 0.0 | 82.6 | 0.0 | 17.4 |
| Example 6 | Zinc carboxylate salt 6 | 33.3 | 0.0 | 6.8 | 59.9 |
| Comparative Example 1 | Zinc carboxylate salt 7 | 100.0 | 0.0 | 0.0 | 0.0 |
| Comparative Example 2 | Zinc carboxylate salt 8 | 0.0 | 0.0 | 0.0 | 100.0 |

TABLE 3

| | Ratio of | | | | | |
|---|---|---|---|---|---|---|
| | C8 to C10 | | Metal | | | Rate of |
| | carboxylic | Average | content | Viscosity | Viscosity | change |
| | acids | branching | (% by | at 130° C. | at 50° C. | viscosity |
| | (% by mass) | degree | mass) | (Pa*s) | (Pa*s) | (%) |
| Zinc carboxylate salt 1 | 95.6 | 1.5 | 16.8 | 2.0 | 1730.0 | 99.8 |
| Zinc carboxylate salt 2 | 95.6 | 2.7 | 16.5 | 30.3 | 2120.0 | 98.6 |
| Zinc carboxylate salt 3 | 93.5 | 2.5 | 16.2 | 11.2 | 1270.0 | 99.1 |
| Zinc carboxylate salt 4 | 91.5 | 2.3 | 15.9 | 0.1 | 22.0 | 99.4 |
| Zinc carboxylate salt 5 | 92.9 | 1.9 | 15.9 | 1.5 | 402.0 | 99.6 |
| Zinc carboxylate salt 6 | 99.7 | 1.1 | 16.5 | 2.7 | 9080.0 | 100.0 |
| Zinc carboxylate salt 7 | 99.6 | 3.1 | 17.2 | 11.6 | 120.0 | 90.3 |
| Zinc carboxylate salt 8 | 99.7 | 0.0 | 16.0 | 14400.0 | 10400.0 | 11.1 |

TABLE 4

| | Zine precursor | | | | | Addition | | Before | | |
|---|---|---|---|---|---|---|---|---|---|---|
| | | Average | | | | ratio | Addition | addition | Reaction | Reaction |
| | Zine carboxy late | branching | | | Other | Zn/In | time | Temperature | Temperature | Time |
| | salt | degree | Solvent | Group VI | additive | Mole ratio | (minutes) | (° C.) | (° C.) | (minutes) |
| Example 7 | Zinc carboxy late | 1.5 | Octadecene | Se | Oleoyl | 15 | 50 | 230 | 300 | 180 |
| | salt 1 | | | | chloride | | | | | |
| Example 8 | Zinc carboxylate | 2.7 | Octadecene | Se, S | Oleoyl | 15 | 50 | 230 | 300 | 180 |
| | salt 2 | | | | chloride | | | | | |
| Example 9 | Zinc carboxylate | 2.5 | Octadecene | Se, S | Oleoyl | 15 | 50 | 230 | 300 | 180 |
| | salt 3 | | | | chloride | | | | | |
| Example 10 | Zinc carboxylate | 2.3 | Octadecene | Se. S | Oleoyl | 15 | 50 | 230 | 300 | 180 |
| | salt 4 | | | | chloride | | | | | |
| Example 11 | Zinc carboxy late | 1.9 | Octadecene | Se, S | Oleoy1 | 15 | 50 | 230 | 300 | 180 |
| | salt 5 | | | | chloride | | | | | |
| Example 12 | Zinc carboxylate | 1.5 | Octadecene | Se, S | Oleoyl | 15 | 50 | 230 | 300 | 180 |
| | salt 1 | | | | chloride | | | | | |
| Example 13 | Zinc carboxylate | 1.1 | Octadecene | Se, S | Oleoyl | 15 | 50 | 230 | 300 | 180 |
| | salt 6 | | | | chloride | | | | | |
| Comparative | Zinc carboxy late | 3.1 | Octadecene | Se, S | Oleoyl | 15 | 50 | 230 | 300 | 180 |
| Example 3 | salt 7 | | | | chloride | | | | | |
| Comparative | Zinc carboxylate | 0.0 | Octadecene | Se, S | Oleoyl | 15 | 50 | 230 | 300 | 180 |
| Example 4 | salt 8 | | | | chloride | | | | | |

TABLE 5

| | | λₘₐₓ | FWHM | |
|---|---|---|---|---|
| | Shell | (nm) | (nm) | QY (%) |
| Example 7 | ZnSe | 630.5 | 36.6 | 91.3 |
| Example 8 | ZnSe/ZnS | 630.3 | 36.4 | 92.2 |
| Example 9 | ZnSe/ZnS | 629.2 | 36.4 | 91.2 |
| Example 10 | ZnSe/ZnS | 626.7 | 35.3 | 91.0 |
| Example 11 | ZnSe/ZnS | 628.8 | 36.5 | 90.3 |
| Example 12 | ZnSe/ZnS | 632.5 | 37.0 | 90.2 |
| Example 13 | ZuSe/ZnS | 629.3 | 36.8 | 90.0 |
| Comparative | ZnSe/ZnS | 628.8 | 37.7 | 87.3 |
| Example 3 | | | | |
| Comparative | ZuSe/ZnS | 631.7 | 36.7 | 85.0 |
| Example 4 | | | | |

1. A zinc carboxylate salt used for production of a semiconductor nanoparticle, wherein
a ratio of carboxylic acids having S to 10 carbon atoms in whole of carboxylic acids that form the zinc carboxylate salt is 80.0% by mass or more, and
an average branching degree of the whole of the carboxylic acids that form the zinc carboxylate salt is in a range of 1.1 to 2.9.

2. The zine carboxylate salt used for production of a semiconductor nanoparticle according to claim 1, wherein the average branching degree of the whole of the carboxylic acids that form the zine carboxylate salt is in a range of 1.3 to 2.7.

3. The zinc carboxylate salt used for production of a semiconductor nanoparticles according to claim 1, wherein the ratio of carboxylic acids having 8 to 10 carbon atoms in the whole of the carboxylic acids that form the zinc carboxylate salt is 85.0% by mass or more.

4. The zinc carboxylate salt for production of a semiconductor nanoparticle according to claim 1, wherein a rate of change in viscosity of the zinc carboxylate salt indicated by the following formula (1) is in a range of 95.0 to 100.0%:
$$\begin{matrix}General formula ( 1 ) & \\ Rate of change in viscosity ( \% ) = ( ( viscosity ( Pa * s ) at 130 °C - viscosity ( Pa * s ) at 50 °C ) / viscosity ( Pa * s ) at 50 °C ) \times 100 & ( 1 )\end{matrix}$$
(wherein the viscosity (Pas) at 130° C. is a value obtained by measuring the zinc carboxylate salt at a temperature of 130° C. by a dynamic viscoelasticity analyzer, and the viscosity at 50° C. is a value obtained by measuring the zine carboxylate salt at a temperature of 50° C. by a dynamic viscoelasticity analyzer).
